(19) United States
(12) Patent Application Publication
(10) Pub. No.: US 2021/0395164 A1
(43) Pub. Date: Dec. 23, 2021

(54) PRODUCTION OF AROMATIC COMPOUNDS FROM HEAVY OIL

(71) Applicant: Saudi Arabian Oil Company, Dhahran (SA)

(72) Inventors: Ki-Hyouk Choi, Dhahran (SA); Ali Al-Nasir, Dhahran (SA); Emad Shafei, Dhahran (SA)

(73) Assignee: Saudi Arabian Oil Company, Dhahran (SA)

(21) Appl. No.: 16/908,491

(22) Filed: Jun. 22, 2020

(51) Int. Cl. C07C 2/06; B01J 3/00; B01J 19/00; B01D 3/06; B01D 5/00; B01D 17/02

(52) U.S. Cl. CPC C07C 2/06; B01J 3/008; B01J 19/0013; B01J 2219/00162; B01D 5/006; B01D 17/02; B01J 2219/00159; B01D 3/06

(57) ABSTRACT

A process to produce aromatic compounds in a heavy oil product stream comprising the steps of separating the depressurized effluent to produce a vapor product stream and a liquid product stream, reducing a temperature of the vapor product stream to produce a cooled vapor product, separating the cooled vapor product to produce a light oil stream, wherein the light oil stream comprises olefins, separating the light oil stream to produce a light oil slip stream and a light stream, mixing the light stream with a water feed stream to produce an olefin-containing water stream, increasing a pressure of the olefin-containing water stream to produce a pressurized water feed, increasing a temperature of the pressurized water feed to produce a hot water feed, wherein a temperature of the hot water feed is greater than 450° C., converting olefins to aromatic compounds in the hot water feed.

### TECHNICAL FIELD

Disclosed are methods for upgrading petroleum. Specifically, disclosed are methods and systems for producing aromatic compounds from heavy oil.

### BACKGROUND

Olefins, including 1-olefins, are useful and valuable chemicals when used as a raw material. For example, 1-olefins can be used as a raw material for the production of linear low density polyethylene (LLDPE), high density polyethylene (HDPE), polyalphaolefin (PAO), linear alkyl benzene (LAB), and linear alkyl benzene sulfonate (LABS). Alpha-olefins for use as a raw material are generally produced as the primary production product, such as in the Ziegler process. Alpha-olefins can be produced in other ways, such as thermal cracking or residue or crude oils, but due to their wide carbon number range, they are not readily or economically separable from the n-paraffins that are also produced.

Reactions in supercritical water can produce significant amounts of olefins, particularly 1-olefins. Alkyl radicals, including alkyl aromatic radicals, formed under thermolysis conditions, can undergo propagation by two paths: hydrogen abstraction and beta-scission. Hydrogen abstraction takes hydrogen from other compounds and the alkyl radicals are converted to alkanes. 1-olefins can be formed through beta-scission, where the alkyl radical cracks to produce an alkyl radical and a 1-olefin. Beta-scission does not require additional molecules. Hydrogen abstraction reactions are suppressed in supercritical water reactions due to the dilution effect of the supercritical water, making it difficult to find hydrogen donor compounds. In contrast, beta-scission of alkyl radical is increased under supercritical water conditions because such donor compounds are not required. Thus, under supercritical water conditions, more 1-olefins tend to be formed than under conventional thermal cracking conditions. However, 1-olefins are unstable under thermolysis conditions, as compared to alkanes, and can be cracked to form radicals that participate in additional reactions. Isomerization of 1-olefins to produce inner olefins, that is olefins where the double bond is at a position other than alpha, through hydrogen abstraction is suppressed due to the dilution effect of supercritical water. For this reason, product oil produced in the presence of supercritical conditions treatment contains significant amounts of 1-olefins with minor amounts of internal olefins. But, such 1-olefins can produce aromatic compounds through radical-mediated cyclization, which is also augmented due to suppressed hydrogen abstraction from dilution of the 1-olefin radicals.

In addition to being difficult to separate from the other fractions, such as paraffins and aromatics, in an upgraded oil, 1-olefins can make the upgraded oil unstable. In fact, olefins in general reduce stability of petroleum-based oil, such as gasoline, diesel, and fuel oil, because those can form gums through oxidation reactions with air. Thus, to improve stability of product oil from supercritical water treatment, 1-olefins must be converted to more stable chemicals. For example, one way to improve stability, is for olefins to be saturated by hydrotreating processes. However, hydrotreating processes need additional units and hydrogen supply along with catalyst. Thus, a way to reduce olefin content in the absence of hydrotreating is desired.

### SUMMARY

Disclosed are methods for upgrading petroleum. Specifically, disclosed are methods and systems for producing aromatic compounds from heavy oil.

In a first aspect, a process to produce aromatic compounds in a heavy oil product stream is provided. The process includes the steps of introducing a depressurized effluent to a flash column, separating the depressurized effluent in the flash column to produce a vapor product stream and a liquid product stream, introducing a vapor product stream to a vapor cooler, reducing a temperature of the vapor product stream in the vapor cooler to produce a cooled vapor product, introducing the cooled vapor product to a three-phase separator, separating the cooled vapor product in the three-phase separator to produce a light oil stream, wherein the light oil stream includes olefins, separating the light oil stream in a splitter to produce a light oil slip stream and a light stream, introducing the light stream to a light stream pump, increasing a pressure of the light stream in the light stream pump to produce a pressurized light stream, mixing the pressurized light stream with a pressurized water feed in a water mixer to produce an olefin-containing water stream, increasing a temperature of the olefin-containing water stream in a water pre-heater to produce a hot water feed, wherein a temperature of the hot water feed is greater than 450° C., converting olefins to aromatic compounds in the hot water feed, introducing the liquid product stream to an oil-water separator, separating the liquid product stream in the oil-water separator to produce a water stream and a heavy stream, and mixing the heavy stream with the light oil slip stream to produce the heavy oil product stream.

In certain aspects, the process further includes the steps of introducing a hydrocarbon feed to an oil pump, increasing a pressure of the hydrocarbon feed in the oil pump to produce a pressurized hydrocarbon feed, introducing the pressurized hydrocarbon feed to an oil pre-heater, increasing a temperature of the pressurized hydrocarbon feed in the oil pre-heater to produce a hot hydrocarbon feed, introducing a water feed stream to a water pump, increasing a pressure of the water feed stream to produce the pressurized water feed, introducing the hot water feed to a mixer, introducing the hot hydrocarbon feed to a mixer, mixing the hot olefin-containing water feed and the hot hydrocarbon feed in the mixer to produce a mixed feed stream, introducing the mixed feed stream to a supercritical water reactor, maintaining the supercritical water reactor at supercritical conditions to produce a reaction effluent, introducing the reaction effluent to a cooling device, reducing a temperature of the reaction effluent in the cooling device to produce a cooled effluent, introducing the cooled effluent to a depressurizing device, and reducing a pressure of the cooled effluent in the depressurizing device to produce the depressurized effluent. In certain aspects, the temperature of the hot water feed is between 450° C. and 600° C. In certain aspects, an amount of water in the liquid product stream is less than 1 wt %. In certain aspects, the light oil stream further includes aromatic compounds and paraffins. In certain aspects, an amount of olefins in the light oil stream is greater than 25 wt %. In certain aspects, an amount of coke in the heavy oil product stream is less than 1 wt %. In certain aspects, a weight ratio of the light stream to the light oil stream is in the range between 5:5 and 9:1. In certain aspects, a temperature of the supercritical water reactor is between 380° C. and 450° C.

In a second aspect, a system for producing aromatic compounds in a heavy oil product stream is provided. The system includes a flash column configured to separate a depressurized effluent to produce a vapor product stream and a liquid product stream, a vapor cooler fluidly connected to a flash column, the flash column configured to reduce a temperature of the vapor product stream to produce a cooled vapor product, a three-phase separator fluidly connected to a vapor cooler, the three-phase separator configured to separate the cooled vapor product to produce a light oil stream, wherein the light oil stream includes olefins, a splitter fluidly connected to the three-phase separator, the splitter configured to separate the light oil stream to produce a light oil slip stream and a light stream, a light stream pump fluidly connected to the splitter, the light stream pump configured to increase a pressure of the light stream to produce a pressurized light stream, a water mixer fluidly connected to the light stream pump, the water mixer configured to mix the pressurized light stream with a pressurized water feed to produce an olefin-containing water stream, a water pre-heater fluidly connected to the water pump, the water pre-heater configured to increase a temperature of the pressurized water feed to produce a hot water feed, wherein a temperature of the hot water feed is greater than 450° C. such that olefins in the olefin-containing water stream are converted to aromatic compounds, an oil-water separator fluidly connected to the flash column, the oil-water separator configured to separate the liquid product stream to produce a water stream and a heavy stream, and an oil mixer fluidly connected to the oil-water separator and the splitter, the oil mixer configured to mix the heavy stream with the light oil slip stream to produce the heavy oil product stream.

In certain aspect, the system further includes an oil pump configured to increase a pressure of a hydrocarbon feed to produce a pressurized hydrocarbon feed, an oil pre-heater fluidly connected to the oil pump, the oil pre-heater configured to increase a temperature of the pressurized hydrocarbon feed to produce a hot hydrocarbon feed, a water pump configured to increase a pressure of a water feed stream to produce the pressurized water feed, a mixer fluidly connected to the oil pre-heater and the water pre-heater, the mixer configured to mix the hot water feed and the hot hydrocarbon feed to produce a mixed feed stream, a supercritical water reactor fluidly connected to the mixer, the supercritical water reactor configured to maintain conversion reactions to produce a reaction effluent, a cooling device fluidly connected to the supercritical water reactor, the cooling device configured to reduce a temperature of the reaction effluent to produce a cooled effluent, and a depressurizing device fluidly connected to the cooling device, the depressurizing device configured to reduce a pressure of the cooled effluent to produce the depressurized effluent. In certain aspects, a ratio of length to diameter of the flash column is in the range between 5 and 15. In certain aspects, the supercritical water reactor includes one or more tubular reactors with a length to diameter ratio of greater than 100 oriented vertically.

### BRIEF DESCRIPTION OF THE DRAWINGS

These and other features, aspects, and advantages of the scope will become better understood with regard to the following descriptions, claims, and accompanying drawings. It is to be noted, however, that the drawings illustrate only several embodiments and are therefore not to be considered limiting of the scope as it can admit to other equally effective embodiments.

FIG. 1 provides a process diagram of an embodiment of the process.

FIG. 2 is a graphical image of GC-MS data from the example.

### DETAILED DESCRIPTION

While the scope will be described with several embodiments, it is understood that one of ordinary skill in the relevant art will appreciate that many examples, variations and alterations to the apparatus and methods described here are within the scope and spirit. Accordingly, the embodiments described are set forth without any loss of generality, and without imposing limitations, on the embodiments. Those of skill in the art understand that the scope includes all possible combinations and uses of particular features described in the specification.

Described here are processes and systems of increasing the amount of aromatic compounds in an upgraded oil stream. Advantageously, the processes and systems described here can reduce or substantially remove olefins from the heavy oil stream and at the same time increase the amount of aromatic compounds, which can improve the stability of the heavy oil. Advantageously, the systems and processes described here improve the stability of the upgraded oil without the need for stabilization processes or stabilization additives, such as additives to prevent gum formation, such as hindered phenols or phenylene diamines. Advantageously, recycling the light oil stream to the water stream can convert the olefins into aromatic compounds increasing the amount of aromatic compounds in the upgraded oil stream.

As used throughout, “external supply of hydrogen” refers to hydrogen, in gas (H₂) or liquid form, supplied as a feed or part of a feed to a unit in the system. External supply of hydrogen does not encompass hydrogen present in the petroleum feedstock.

As used throughout, “external supply of catalyst” refers to a catalyst added to a unit as either a part of the feed to the unit or present in the empty unit, for example as a catalyst bed. External supply of catalyst does not encompass compounds that could have a catalytic effect and are part of the petroleum feedstock or produced through reactions within the units of the system.

As used throughout, “in the absence of” means does not contain, does not include, does not comprise, is without, or does not occur.

As used throughout, “coke” refers to the toluene insoluble material present in petroleum.

As used throughout, “asphaltene fraction” or “asphaltenic fraction” refers to the n-heptane-insoluble, but toluene-soluble fraction of hydrocarbons.

As used throughout, “upgrade” means to increase the API gravity, decrease the amount of impurities, such as sulfur, nitrogen, and metals, decrease the amount of asphaltense and increase the amount of the light fraction.

As used throughout, “1-olefins,” “alpha-olefins” or “α-olefins” refers to alkenes having a chemical formula of CₓH₂ₓ, with a double bond at the alpha position. Alpha-olefins can include branched and linear compounds.

As used throughout, T90% refers to a distillation temperature where 90 volume percent (vol %) of oil can be distilled.

As used throughout, “stability” or “stable” refers to storage stability, which can be assessed by standard methods captured in ASTM D7060, ASTM D2274, and ASTM D381.

It is known in the art that hydrocarbon reactions in supercritical water upgrade heavy oil to produce products that have lighter fractions. Supercritical water has unique properties making it suitable for use as a petroleum reaction medium where the reaction objectives include upgrading reactions, desulfurization reactions and demetallization reactions, where supercritical water acts as both a hydrogen source and a solvent (diluent). Supercritical water is water greater than the critical temperature of water and greater than the critical pressure of water. The critical temperature of water is 373.946 deg C. The critical pressure of water is 22.06 megapascals (MPa). Without being bound to a particular theory, it is understood that the basic reaction mechanism of supercritical water mediated petroleum processes is the same as a radical reaction mechanism. Thermal energy creates radicals through chemical bond breakage. Supercritical water, acting as a diluent, creates a “cage effect” by surrounding radicals. The radicals surrounded by water molecules cannot react easily with each other, and thus, intermolecular reactions that contribute to coke formation are suppressed. The cage effect suppresses coke formation by limiting inter-radical reactions compared to conventional thermal cracking processes, such as delayed coker. Hydrogen from the water molecules can be transferred to the hydrocarbons through direct transfer or through indirect transfer, such as the water gas shift reaction. While, supercritical water facilitates hydrogen transfer between molecules, it is inevitable to produce unsaturated hydrocarbons due to the limited amount of available hydrogen. Unsaturated carbon-carbon bonds in the upgraded oil can be distributed through the whole range of boiling points. Olefins, as a representative unsaturated hydrocarbon, are valuable chemicals, but low stability can cause many problems such as gum formation when exposed to air. Thus, it is common practice in modern refinery to saturate olefins with hydrogen in the presence of catalyst. Thermal cracking of a paraffin feed can produce paraffins and olefins having reduced numbers of carbons compared to the paraffin feed. Thermal cracking to produce olefins occurs in part due to the limited availability of hydrogen in the supercritical water reactor. The olefins can be converted to aromatic compounds, including polyaromatic hydrocarbons, by cyclization at temperatures greater than 450° C. The relative amount of paraffins and olefins and the distribution of carbon numbers in the product strongly depends on the phase where the thermal cracking occurs. In the liquid phase, faster hydrogen transfer between molecules occurs due to the high density creating closer distances between the molecules which makes hydrogen transfer between molecules easier and faster. In the gas phase, methane, ethane, and other light paraffin gases are produced and consumer large amounts of hydrogen. Thus, the liquid phase facilitates the formation of more paraffins in the liquid phase product as compared to gas-phase cracking. Additionally, liquid phase cracking shows generally even distribution of the carbon numbers of the product while gas phase cracking has more light paraffins and olefins in the product.

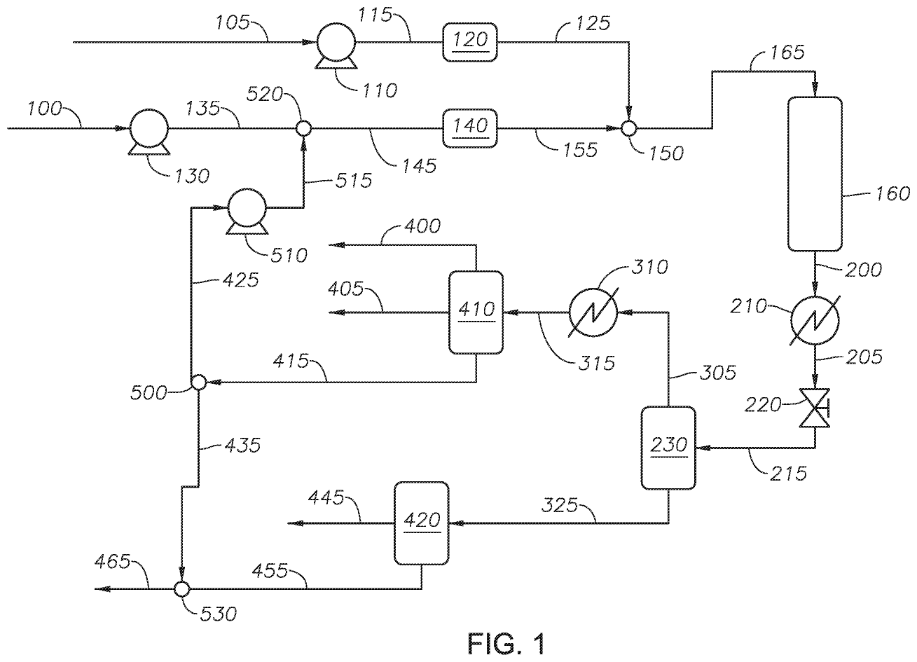

Referring to FIG. 1, an embodiment of the process to produce heavy oil product is provided. Hydrocarbon feed 105 is transferred to oil pre-heater 120 through oil pump 110. Hydrocarbon feed 105 can be any source of petroleum-based hydrocarbons having an API gravity of less than 41, a T90% greater than 1000° F. (537° C.), and an aromaticity less than 0.2 as measured by proton NMR(fa). The aromaticity provides a measure of the paraffin target. Maintaining an aromaticity below 0.2 can ensure the hydrocarbon feed contains sufficient paraffins and alkyl chain hydrocarbons to generate olefins, including 1-olefins. Examples of petroleum-based hydrocarbon sources include whole range crude oil, distilled crude oil, residue oil, topped crude oil, product streams from oil refineries, product streams from steam cracking processes, liquefied coals, liquid products recovered from oil or tar sands, bitumen, oil shale, asphaltene, biomass hydrocarbons, liquid product from gas-to-liquid processes, and combinations of the same. The exact source of hydrocarbon feed 105 can be selected to have the properties described. Hydrocarbon feed 105 can be heated to a temperature that can allow hydrocarbon feed 105 to flow. The temperature of hydrocarbon feed 105 can be greater than ambient temperature, alternately less than 150° C., and alternately between ambient temperature and 150° C.

Oil pump 110 increases the pressure of hydrocarbon feed 105 to produce pressurized hydrocarbon feed 115. Oil pump 110 can be any type of pump capable of increasing the pressure of hydrocarbon feed 105. Examples of oil pump 110 include a diaphragm metering pump. Hydrocarbon feed 105 has a feed pressure. The feed pressure of hydrocarbon feed 105 is a pressure greater than the critical pressure of water, alternately a pressure greater than about 23 MPa, and alternately a pressure between about 23 MPa and about 30 MPa. In at least one embodiment, the feedstock pressure is about 24 MPa.

Oil pre-heater 120 increases the temperature of pressurized hydrocarbon feed 115 to produce hot hydrocarbon feed 125. Oil pre-heater 120 can be any type of heating device that can increase the temperature of pressurized hydrocarbon feed 115. Examples of oil pre-heater 120 can include an electric heater, a gas-fired heater, a steam heater, and a heat exchanger. Oil pre-heater 120 heats pressurized hydrocarbon feed 115 to a feed temperature. The feed temperature of hot hydrocarbon feed 125 is a temperature equal to or less than 250° C., alternately a temperature less than 200° C., alternately a temperature between about 30° C. and 250° C., alternately a temperature between 30° C. and 200° C., alternately a temperature less than 150° C., and alternately a temperature between 50° C. and 150° C. In at least one embodiment, the feed temperature is 150° C. Maintaining a temperature of less than 250° C. reduces or eliminates the production of coke in oil pre-heater 120. The temperature of hot hydrocarbon feed 125 and the amount of heat added in oil pre-heater 120 can be based on the source of hydrocarbon feed 105 and the temperature of hydrocarbon feed 105.

Water feed stream 100 is introduced to water mixer 520 through water pump 130.

Water feed stream 100 can be any source of is demineralized water with a conductivity of less than 1.0 microSiemens per centimeter (μS/cm²), alternately a conductivity of less than 0.1 μS/cm², and alternately a conductivity of less than 0.1 μS/cm². The sodium content of water feed stream 100 can be less than 5 micrograms per liter (μg/l) and alternately less than 1 μg/l. The chloride content of water feed stream 100 can be less than 5 μg/l and alternately less than 1 μg/l. The silica content of water feed stream 100 can be less than 3 μg/l.

Water feed stream 100 can be fed to water pump 130 to create pressurized water feed 135. Water pump 130 can be any type of pump capable of increasing the pressure of water feed stream 100. Examples of pumps suitable for use as water pump 130 include a diaphragm metering pump. Pressurized water feed 135 has a water pressure. The water pressure of pressurized water feed 135 can be a pressure greater than the critical pressure of water, alternately a pressure greater than about 23 MPa, and alternately a pressure between about 23 MPa and about 30 MPa. In at least one embodiment, the water pressure is about 24 MPa.

Pressurized water feed 135 can be mixed with pressurized light stream 515 in water mixer 520 to produced olefin-containing water stream 145. Water mixer 520 an be any type of mixer capable of mixing a water stream and a hydrocarbon-containing stream. Olefin-containing water stream 145 can be fed to water pre-heater 140 to create hot water feed 155. Olefin-containing water stream 145 can include olefins and water. Olefins in olefin-containing water stream 145 can include 1-olefins.

Water pre-heater 140 heats olefin-containing water stream 145 to a water temperature to produce hot water feed 155. Water pre-heater 140 can be any type of heating device that can increase the temperature of olefin-containing water stream 145. Examples of water pre-heater 140 can include a gas-fired heater, an electric heater, and a heat exchanger. The water temperature of olefin-containing water stream 145 is a temperature greater than the critical temperature of water, alternately greater than 450° C., alternately greater than 475° C., alternately between about 450° C. and about 600° C., alternately between 450° C. and 550° C., alternately between 450° C. and 500° C., alternately between about 475° C. and about 600° C., alternately between 475° C. and 550° C., alternately between 475° C. and 500° C., alternately between 500° C. and 600° C., and alternately between 500° C. and 550° C. In at least one embodiment, the temperature of water pre-heater 140 is between 500° C. and 550° C. Hot water feed 155 contains supercritical water. The upper limit of the water temperature is constrained by the rating of the physical aspects of the process, such as pipes, flanges, and other connection pieces. For example, for 316 stainless steel, the maximum temperature at high pressure is recommended to be 649° C. Temperatures less than 600° C. are a practical range within the physical constraints of the pipelines. In at least one embodiment, hot water feed 155 is at a temperature between 500° C. and 550° C. The residence time in water pre-heater 140 can be between 10 seconds and 10 minutes and alternately between 30 seconds and 3 minutes. The residence time can be calculated by assuming the density of the fluid in the water pre-heater is the density of water at the operating conditions of the water pre-heater.

Due to the temperature in water pre-heater 140, the olefins in olefin-containing water stream 145 can be converted to aromatic compounds in water pre-heater 140 such that hot water feed 155 can contain water, aromatic compounds, olefins, and combinations of the same. The aromatic compounds in olefin-containing water stream 145 that are recycled as part of light stream 425 are relatively stable and therefore reactions involving those aromatic compounds are minimized or eliminated. Paraffins in olefin-containing water stream 145 that are recycled as part of light stream 425 can be cracked to produce olefins and paraffins. However, due to the low concentration of paraffins in olefin-containing water stream 145 and the cage effect due to the water, bimolecular reactions involving the paraffin radicals are significantly suppressed and alternately eliminated. Thus, alkylation and condensation of aromatics into larger ring aromatics does not occur at a detectable level. Converting the olefins upstream of supercritical water reactor 160 means the temperature in supercritical water reactor 160 can be maintained at less than 450° C. Olefin-containing water stream 145 is in the absence of asphaltenic fraction such that hot water feed 155 is in the absence of coke.

Hot water feed 155 contains supercritical water at conditions greater than the critical temperature of water and critical pressure of water.

The internal volume of the piping, fittings, and other conduits between the source of hydrocarbon feed 105 and mixer 150 and the source of water feed stream 100 and mixer 150 can be designed to provide a residence time of the fluids in the range between 10 seconds and 10 minutes and alternately between 30 seconds and 3 minutes. The residence time is calculated by assuming the density of the fluid is the density of water at the operating conditions of the fluid.

Water feed stream 100 and hydrocarbon feed 105 are pressurized and heated separately. In at least one embodiment, the temperature difference between hot hydrocarbon feed 125 and hot water feed 155 is greater than 300° C. Without being bound to a particular theory, a temperature difference between hot hydrocarbon feed 125 and hot water feed 155 of greater than 300° C. is believed to increase the mixing of the petroleum-based hydrocarbons present in hot hydrocarbon feed 125 with the supercritical water in hot water feed 155 in mixer 150. Regardless of the order of mixing, hydrocarbon feed 105 is not heated to greater than 250° C. until after having been mixed with water feed stream 100 to avoid the production of coke.

Hot water feed 155 and hot hydrocarbon feed 125 are fed to mixer 150 to produce mixed feed stream 165. Mixer 150 can include any mixer capable of mixing a petroleum-based hydrocarbon stream and a supercritical water stream. Examples of mixers suitable for use as mixer 150 include static mixers, a vessel with an internal agitator, tee fittings, ultrasonic mixers, capillary mixers, and any other type of mixer known in the art. Without being bound to a particular theory, supercritical water and hydrocarbons do not instantaneously mix on contact, but require sustained mixing before a well-mixed or thoroughly mixed stream can be developed. A well-mixed stream facilitates the cage-effect of the supercritical water on the hydrocarbons.

The ratio of the volumetric flow rate of hydrocarbon feed to water entering supercritical water reactor 160 at standard ambient temperature and pressure (SATP) is between about 1:10 and about 1:0.1 vol/vol, and alternately between about 1:1 and 1:5. In at least one embodiment, the ratio of the volumetric flow rate of hot hydrocarbon feed 125 to the volumetric flow rate of hot water feed 155 entering supercritical water reactor 160 is in the range of 1:1 to 1:5 vol/vol at SATP.

Having a well-mixed mixed feed stream 165 can increase the conversion of hydrocarbons in the reactor. The temperature of mixed feed stream 165 depends on the water temperature of hot water feed 155, the feed temperature of hot hydrocarbon feed 125, and the ratio of hot water feed 155 to hydrocarbon feed 125. The temperature of mixed feed stream 165 can be between 270° C. and 500° C., alternately between 300° C. and 500° C., and alternately between 300° C. and 374° C. In at least one embodiment, the temperature of mixed feed stream 165 is greater than 300° C. The pressure of mixed feed stream 165 depends on the water pressure of hot water feed 155 and the feed pressure of hot hydrocarbon feed 125. The pressure of mixed feed stream 165 can be greater than 22 MPa.

Mixed feed stream 165 is introduced to supercritical water reactor 160 to produce reactor effluent 200. In at least one embodiment, mixed feed stream 165 passes from mixer 150 to supercritical water reactor 160 in the absence of an additional heating step. In at least one embodiment, mixed feed stream 165 passes from mixer 150 to supercritical water reactor 160 in the absence of an additional heating step, but through piping with thermal insulation to maintain the temperature.

Supercritical water reactor 160 is operated at a temperature greater than the critical temperature of water, alternately between about 374° C. and about 500° C., alternately between about 380° C. and about 450° C., alternately between about 390° C. and about 450° C., alternately between about 400° C. and about 450° C., alternately between about 400° C. and about 440° C., alternately between about 410° C. and about 440° C., and alternately between 420° C. and about 440° C. In at least one embodiment, the temperature in supercritical water reactor 160 is between 420° C. and about 440° C. The temperature in supercritical water reactor 160 can be maintained by an external heater, an internal heater, through insulation surrounding supercritical water reactor 160 or combinations of the same. The external heater can be any type of heater known in the art, including an electric heater and a fired heater. Advantageously, maintaining the temperature in supercritical water reactor 160 at less than 450° C. can minimize the conversion of the asphaltenic fraction to solid coke. Coke can be formed by thermal condensation of heavy molecules, such as the asphaltenic fraction. Advantageously, maintaining the temperature in supercritical water reactor 160 below 450° C. means that special materials of construction for supercritical water reactor 160 are not required and standard materials can be used.

Supercritical water reactor 160 is at a pressure greater than the critical pressure of water, alternately greater than about 220 bar (22 MPa), alternately between 220 bar (22 MPa) and 300 bar (30 MPa), and alternately between 250 bar (25 MPa) and 280 bar (28 MPa). Supercritical water reactor 160 can be any type of reactor capable of supporting conversion reactions in the presence of water at supercritical conditions. Supercritical water reactor 160 is in the absence of an external supply of catalyst. Supercritical water reactor 160 is in the absence of an external supply of hydrogen. Supercritical water reactor 160 can be a tubular type reactor with ratio of length to diameter of greater than 100, a vessel type reactor with a length to diameter ratio of less than 100, continuous stirred tank reactor, and combinations of the same. In at least one embodiment, supercritical water reactor 160 is one or more tubular reactors with a length to diameter ratio of each reactor of greater than 100. The one or more tubular reactors can be oriented horizontally, vertically, sloped at an angle between horizontal and vertical, and combinations of the same. In at least one embodiment, supercritical water reactor 160 is one or more tubular reactors with a length to diameter ratio of greater than 100 oriented vertically. The vertically oriented one or more tubular reactors can be upflow, downflow, or combination of the same. In at least one embodiment, supercritical water reactor 160 is one or more tubular reactors with a length to diameter ratio of greater than 100 oriented vertically with downflow. The internal volume of supercritical water reactor 160 can be designed such that mixed feed stream 165 has the desired residence time. The residence time of mixed feed stream 165 in supercritical water reactor 160 can be greater than about 30 seconds, alternately between about 30 seconds and about 60 minutes, alternately between about 30 seconds and 15 minutes, alternately between about 1 minute and about 60 minutes, and alternately between about 1 minute and about 15 minutes. The residence time in supercritical water reactor 160 can be calculated by assuming the density of the internal fluid is the density of water at the operating conditions in supercritical water reactor 160. Conversion reactions can occur in supercritical water reactor 160. Exemplary conversion reactions include cracking, isomerization, alkylation, dimerization, aromatization, cyclization, desulfurization, denitrogenation, demetallization, and combinations thereof. Reactor effluent 200 can include olefins, paraffins, aromatics, naphthenes, asphaltenes, water, and combinations of the same. The olefins in reactor effluent 200 can include 1-olefins.

Reactor effluent 200 is fed to cooling device 210 to produce cooled effluent 205. Cooling device 210 can be any unit capable of reducing the temperature of reactor effluent 200. In at least one embodiment, cooling device 210 is a heat exchanger. Cooled effluent 205 is at a temperature at or less than the critical temperature of water and alternately in the range between 200° C. and 350° C. The temperature of cooled effluent 205 can be based on the pressure in flash column 230 and the desired properties of the separated streams. In at least one embodiment, cooled effluent 205 is at a temperature 200° C. and 350° C. The temperature control by cooling device 210 facilitates separation of depressurized effluent 215 in flash column 230 without the need for further heating.

Cooled effluent 205 passes through depressurizing device 220 to produce depressurized effluent 215. Depressurizing device 220 can be any pressure regulating device capable of reducing the pressure of a fluid. Examples of pressure regulating devices that can be used as depressurizing device 220 include pressure control valves, capillary elements, and back pressure regulators. In at least one embodiment, depressurizing device 220 can be a back pressure regulator. The pressure of depressurized effluent 215 can be in the range between about 2 bar (0.2 MPa) and 50 bar (5 MPa) and alternately between about 10 bar (1 MPa) and 20 bar (2 MPa).

The piping through which depressurized effluent 215 passes from depressurizing device 220 to flash column 230 can have a heater (not shown) to adjust the temperature depressurizing effluent 215 upstream of flash column 230. Heaters suitable for use on the piping connected depressurizing device 220 and flash column 230 can include an electric heater, a steam heater, heat traced insulation, and combinations of the same. The temperature of depressurized effluent 215 can be in the range between 150° C. and 270° C.

Depressurized effluent 215 is fed to flash column 230. Flash column 230 separates depressurized effluent 215 into vapor product stream 305 and liquid product stream 325. Flash column 230 can be a simple fractionator, such as a flash drum. The temperature and pressure in flash column 230 impact the separation of components in depressurized effluent 215. The temperature and pressure in flash column 230 can be adjusted based on the amount of water in liquid product stream 325 and the desired properties of light oil stream 415. The amount of water in liquid product stream 325 can be less than about 1 wt % and alternately less than about 0.5 wt %. In at least one embodiment, the amount of water in liquid product stream 325 is less than or equal to about 0.5 wt %. Light oil stream 415 can have a T90% less than 650° F. (343° C.) as measured by ASTM D2887. In at least one embodiment, light oil stream 415 can have a T95% of 200° C. Flash column 230 can include a heater to adjust the temperature to between 150° C. and 270° C. The internal volume of flash column 230 can be calculated based on the total volumetric flow rate of hydrocarbons and water at SATP. Flash column 230 can be vertically oriented. The ratio of length to diameter of flash column 230 can be in the range between 5 and 15. The inlet port in flash column 230 through which depressurized effluent 215 flows can be positioned at a point near the top between 10% and 40% of the total length. For example, if the total length of flash column 230 is 2000 mm, the inlet port can be positioned at a distance of 600 mm as measured from the top.

The temperature of vapor product stream 305 can be reduced in vapor cooler 310 to produce cooled vapor product 315. Vapor cooler 310 can be any type of cooler capable of reducing a temperature of cooled vapor product 315 to condense all or part of the components in vapor product stream 305.

The pressure of vapor product stream 305 and or cooled vapor product 315 can be adjusted upstream or downstream of vapor cooler 310 as needed to achieve the desired separation in three-phase separator 410.

Cooled vapor product 315 can be introduced to three-phase separator 410. Due to the absence of heavy compounds in vapor product stream 305 three-phase separator 410 can be in the absence of a demulsifier. The absence of heavy compounds in vapor product stream 305 means the heavy compounds are not present to act as a surfactant in the emulsion. Three-phase separator 410 can be any type of separator capable of separating the components in cooled vapor product 315. Cooled vapor product 315 can be separated in three-phase separator 410 to produce gases stream 400, condensed water 405, and light oil stream 415. Gases stream 400 can contain methane, ethane, ethylene, propane, propylene, butane, butylene, hydrogen sulfide, carbon monoxide, carbon dioxide, and combinations of the same. Condensed water 405 can contain water condensed in three-phase separator 410. Condensed water 405 can contain water, dissolved organic compounds, and combinations of the same. Condensed water 405 can contain an amount of total organic carbon in the range between 10 parts-per-million by weight (wt ppm) and 2,000 wt ppm, alternately between 100 wt ppm and 1,000 wt ppm, alternately between 200 wt ppm and 600 wt ppm, and alternately between 300 wt ppm and 500 wt ppm. In at least one embodiment, the amount of total organic carbon is in the range between 300 wt ppm and 500 wt ppm. Condensed water 405 can contain the majority of the water in reactor effluent 200. Condensed water 405 can contain greater than 90 wt % of the water in mixed feed stream 165. Light oil stream 415 can contain olefins, aromatic compounds, paraffins, and combinations of the same. The olefins in light oil stream 415 can include 1-olefins. The conditions in three-phase separator 410 can be adjusted based on the desired concentration of olefins in light oil stream 415. The concentration of olefins in light oil stream 415 can be greater than about 25 wt %, alternately between about 25 wt % and about 40 wt %, and alternately about 40 wt %. The concentration of 1-olefins in light oil stream 415 can be greater than 50 wt % of the total olefins in light oil stream 415, alternately greater than 75 wt % of the total olefins in light oil stream 415, alternately between about 50 wt % and about 99 wt % of the total olefins in light oil stream 415, and alternately between about 75 wt % and about 99 wt % of the total olefins in light oil stream 415. Light oil stream 415 can contain less than 0.3 wt % water, alternately less than 0.1 wt % water, alternately less than a detectable amount of water, and alternately can be in the absence of water. Light oil stream 415 is in the absence of asphaltenic fraction. Advantageously, recycling a portion of olefins in light oil stream 415 can increase the stability of produced oil in heavy oil stream 455. Separating light oil stream 415 provides the ability to control the amount of olefins in heavy oil product stream 465, which would not be possible using methods for separating the water and olefins from other hydrocarbons. The hydrocarbons in light oil stream 415 are not readily miscible in water, thus mixing condensed water 405 and light oil stream 415 would result in two phase flow due to phase separation, which is difficult to pump.

Light oil stream 415 can be split in splitter 500 to produce light stream 425 and light oil slip stream 435. The weight ratio of light stream 425 to light oil slip stream 435 can be in the range between 5:5 to 9:1 (wt/wt). Light stream 425 and light oil slip stream 435 have the same composition and properties as light oil stream 415 upstream of splitter 500.

Light stream 425 can be introduced to light stream pump 510. The pressure of light stream 425 can be increased in light stream pump 510 to produce pressurized light stream 515. Examples of pump suitable for use as light stream pump 510 can include a diaphragm metering pump. Pressurized light stream 515 can be at a pressure greater than the critical pressure of water, alternately a pressure greater than about 23 MPa, and alternately a pressure between about 23 MPa and about 30 MPa. In at least one embodiment, the water pressure is about 24 MPa. In at least one embodiment, the pressure of pressurized light stream 515 is the same as the water pressure of pressurized water feed 135. Pressurized light stream 515 can be introduced to water mixer 520.

Liquid product stream 325 can be introduced to oil-water separator 420. Liquid product stream 325 can be separated in oil-water separator 420 to produce water stream 445 and heavy oil stream 455. Heavy oil stream 455 can include hydrocarbons having boiling points greater than the boiling points of the hydrocarbons in light stream 425, water, and combinations of the same. The hydrocarbons in heavy oil stream 455 can include paraffins, olefins, aromatics, naphthenes, asphaltenes, resins, coke, and combinations of the same. The amount of coke in heavy oil stream 455 can be less than 1 wt %. The amount of water in heavy oil stream 455 can be between about 0.05 wt % and about 3 wt % and alternately between about 0.1 wt % and 1 wt %. Produced water stream 445 can contain water and dissolved organic compounds.

Heavy oil stream 455 can be mixed with light oil slip stream 435 in oil mixer 530 to produce heavy oil product stream 465. Heavy oil product stream 465 can be in the absence of coke and alternately can contain less than 1 wt % coke. The concentration of aromatics in heavy oil product stream 465 can depend on the aromatic concentration of hydrocarbon feed 105, the operating conditions in supercritical water reactor 160, and the conditions in oil-water separator 420. The concentration of aromatics in heavy oil product stream 465 can be in the range from 5 wt % to 95 wt % and alternately between 25 wt % and 75 wt %. The concentration of olefins in heavy oil product stream 465 can be less than 1 wt %, alternately less than 0.8 wt %, alternately less than 0.5 wt %, and alternately less than 0.2 wt %.

The temperature and pressure of liquid product stream 325 can be adjusted upstream of oil-water separator 420 as needed to achieve the desired separation in oil-water separator 420. Any known equipment that can reduce temperature or reduce pressure can be used to adjust the conditions of liquid product stream 325. Heavy oil product stream 465 can be treated to produce an aromatics product stream and a naphthalene stream. Additional treatments can include distillation and aromatic extraction.

### EXAMPLES

Example 1. Example 1 was a pilot plant test run of the process to produce aromatic compounds. Hydrocarbon feed 105 was an atmospheric residue having the properties shown in Table 1 and in the absence of olefins as confirmed by Proton-NMR. Water feed stream 100 was a demineralized water having a conductivity of 0.056 μS/cm. The flow rate of hydrocarbon feed 105 was 70 kg/hr at SATP. The flow rate of water feed stream 100 was 70 kg/hr at SATP. Hot hydrocarbon feed 125 was at a temperature of 250° C. Water pre-heater 140 was a coiled pipe with a length of 35 meters, an outer diameter of 38.1 mm, and wall thickness of 5 mm. The residence time in water pre-heater was 1.8 minutes. The temperature of hot water feed 155 was 520° C. Supercritical water reactor 160 consisted of five vertically oriented pipes in series, where each pipe was 4 meters long with an inner diameter of 40 mm. The flow direction was downflow in reactors one, three, and five and upflow in reactors two and four. The temperature in supercritical water reactor 160 was regulated by controlling the temperature of reactor effluent 200 to be 448° C.

TABLE 1
Stream properties for Example 1.

| | | | | Heavy Oil |
|---|---|---|---|---|
| | Hydrocarbon | Light Oil | Heavy Oil | Product |
| Property | Feed 105 | Stream 415 | Stream 455 | Stream 465 |
| API | 13.7 | 43.3 | 15.2 | 17.0 |
| Sulfur, | 3.8 | 0.87 | 3.7 | 3.5 |
| wt % | | | | |
| MCR, | 15.5 | — | 3.9 | 3.7 |
| wt % | | | | |
| Asphaltene, | 4.9 | — | 1.2 | 1.1 |
| wt % | | | | |
| Viscosity at | 596 | 0.9 | 171 | 97 |
| 121° F., cSt | | | | |

| Distillation | ASTM | ASTM | ASTM | ASTM |
|---|---|---|---|---|
| (° C.) | D7169 | D2287 | D 7169 | D 7169 |
| 5% | 372 | 112 | 333 | 207 |
| 10% | 409 | 129 | 374 | 282 |
| 30% | 506 | 170 | 457 | 421 |
| 50% | 585 | 200 | 496 | 491 |
| 70% | 663 | 238 | 579 | 567 |

Reactor effluent 200 was cooled in cooling device 210 to 250° C., where cooling device was a water cooled heat exchanger. Depressurizing device 220 reduced the pressure so that the pressure of depressurized effluent 215 was 13 barg (1.3 MPa). The temperature of depressurized effluent 215 was 230° C. Flash column 230 had an internal volume of 63 liters, an inner diameter of 203 mm, and a length of 2,000 mm. The flash column 230 was vertically oriented with the inlet port located at about 600 mm from the top. Vapor product stream 305 had a flow rate of 83 kg/hr. The flow rate of liquid product stream 325 was 57 kg/hr. Vapor product stream 305 was pressurized and cooled such that cooled vapor product 315 was at a pressure of 2.5 barg (0.25 MPa) and 40° C. before being introduced to three-phase separator 410. Cooled vapor product 315 was separated in three-phase separator to produce gases stream 400 with a flow rate of 0.11 kg/hr, condensed water 405 with a flow rate of 69.9 kg/hr, and light oil stream 415 with a flow rate of 13.0 kg/hr. Light oil stream 415 contained 47.7 wt % 1-olefins, 6.3 wt % aromatic compounds, and the remainder paraffins. Light oil stream 415 contained less than a detectable amount of water. Condensed water 405 contained a total organic content of less than 400 wt ppm.

The pressure of liquid product stream 325 was reduced to 2.5 barg (0.25 MPa) and the temperature was reduced to 80° C. before being introduced to oil-water separator 420. Liquid product stream 325 was separated in oil-water separator 420 to produce water stream 445 with a flow rate of 0.13 kg/hr and heavy oil stream 455 with a flow rate of 56.9 kg/hr. Heavy oil stream 455 contained less than 0.12 wt % water.

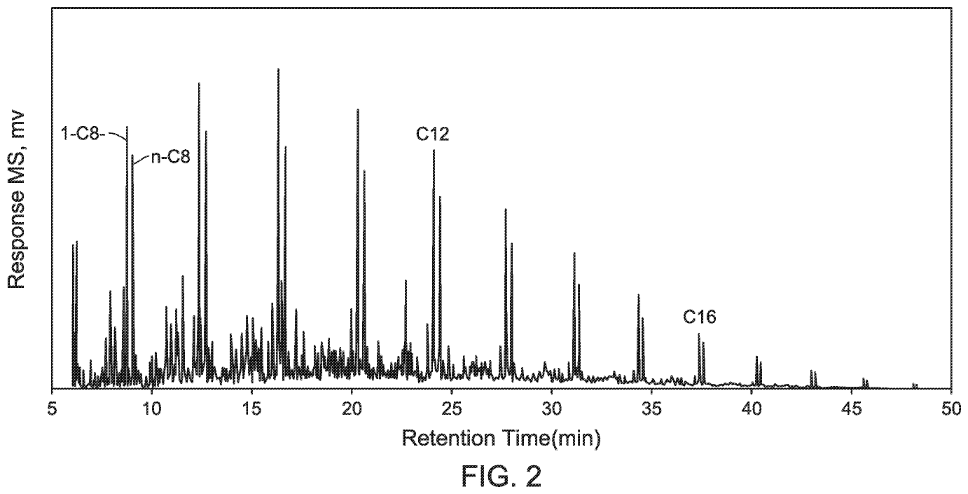

FIG. 2 shows a GC-MS chromatogram of light oil stream 415. The peak noted 1-C8-refers to the peak of 1-olefins and the peak noted n-C8 refers to the peak of n-paraffins. Notably, the 1-olefin peak is taller than the associated n-paraffin peak. This indicates that the process to produce aromatic compounds in a supercritical water reactor produces a significant amount of 1-olefins. The GC-MS data does not indicate the presence of inner olefins confirming that the dilution effect of supercritical water suppresses hydrogen abstraction and thus, isomerization reactions. The GC-MS data does indicate the presence of aromatic compounds. The newly formed 1-olefins were concentrated in the fraction having a boiling point range less than 360° C.

Hot water feed 155 was sampled and analyzed and the concentration of 1-olefins in the hydrocarbon phase was decreased to 12.4 wt % from 47.7 wt % while the aromatic content in the hydrocarbon phase increased to 27.8 wt % from 6.3 wt %. The flow rate of light oil slip stream 435 is 1.5 kg/hr, such that the majority of the flow of light oil stream 415 is contained in light stream 425. Heavy oil stream 455 and light oil slip stream 435 were mixed to produce heavy oil product stream 465. Heavy oil product stream 465 had a concentration of 1-olefins of 3.1 wt %. In contrast, if the entire amount of the light oil stream 415 was mixed with heavy oil stream 455 the concentration of 1-olefins is 10.7 wt %.

Although the present embodiments have been described in detail, it should be understood that various changes, substitutions, and alterations can be made hereupon without departing from the principle and scope. Accordingly, the scope of the embodiments should be determined by the following claims and their appropriate legal equivalents.

There various elements described can be used in combination with all other elements described here unless otherwise indicated.

The singular forms “a”, “an” and “the” include plural referents, unless the context clearly dictates otherwise.

Optional or optionally means that the subsequently described event or circumstances may or may not occur. The description includes instances where the event or circumstance occurs and instances where it does not occur.

Ranges may be expressed here as from about one particular value and to about another particular value or to about another particular value. When such a range is expressed, it is to be understood that another embodiment is from the one particular value to the other particular value, along with all combinations within said range.

Throughout this application, where patents or publications are referenced, the disclosures of these references in their entireties are intended to be incorporated by reference into this application, in order to more fully describe the state of the art, except when these references contradict the statements made here.

As used throughout this application and in the appended claims, the words “comprise,” “has,” and “include” and all grammatical variations are each intended to have an open, non-limiting meaning that does not exclude additional elements or steps.

As used here, terms such as “first” and “second” are arbitrarily assigned and are merely intended to differentiate between two or more components of an apparatus. It is to be understood that the words “first” and “second” serve no other purpose and are not part of the name or description of the component, nor do they necessarily define a relative location or position of the component. Furthermore, it is to be understood that that the mere use of the term “first” and “second” does not require that there be any “third” component, although that possibility is contemplated under the scope of the embodiments.

1. A process to produce aromatic compounds in a heavy oil product stream, the process comprising the steps of:
introducing a depressurized effluent to a flash column;
separating the depressurized effluent in the flash column to produce a vapor product stream and a liquid product stream;
introducing a vapor product stream to a vapor cooler;
reducing a temperature of the vapor product stream in the vapor cooler to produce a cooled vapor product;
introducing the cooled vapor product to a three-phase separator;
separating the cooled vapor product in the three-phase separator to produce a light oil stream, wherein the light oil stream comprises olefins;
separating the light oil stream in a splitter to produce a light oil slip stream and a light stream;
introducing the light stream to a light stream pump;
increasing a pressure of the light stream in the light stream pump to produce a pressurized light stream;
mixing the pressurized light stream with a pressurized water feed in a water mixer to produce an olefin-containing water stream;
increasing a temperature of the olefin-containing water stream in a water pre-heater to produce a hot water feed, wherein a temperature of the hot water feed is greater than 450° C.;
converting 1-olefins to aromatic compounds in the hot water feed;
introducing the liquid product stream to an oil-water separator;
separating the liquid product stream in the oil-water separator to produce a water stream and a heavy stream; and
mixing the heavy stream with the light oil slip stream to produce the heavy oil product stream.

2. The process of claim 1, further comprising the steps of introducing a hydrocarbon feed to an oil pump;
increasing a pressure of the hydrocarbon feed in the oil pump to produce a pressurized hydrocarbon feed;
introducing the pressurized hydrocarbon feed to an oil pre-heater;
increasing a temperature of the pressurized hydrocarbon feed in the oil pre-heater to produce a hot hydrocarbon feed, wherein the hot hydrocarbon feed is at a temperature of less than 250° C.;
introducing a water feed stream to a water pump;
increasing a pressure of the water feed stream in water pump to produce the pressurized water feed;
introducing the hot water feed to a mixer;
introducing the hot hydrocarbon feed to the mixer;
mixing the hot water feed and the hot hydrocarbon feed in the mixer to produce a mixed feed stream;
introducing the mixed feed stream to a supercritical water reactor;
maintaining the supercritical water reactor at supercritical conditions to produce a reaction effluent;
introducing the reaction effluent to a cooling device;
reducing a temperature of the reaction effluent in the cooling device to produce a cooled effluent;
introducing the cooled effluent to a depressurizing device; and
reducing a pressure of the cooled effluent in the depressurizing device to produce the depressurized effluent.

3. The process of claim 1, wherein the temperature of the hot water feed is between 450° C. and 600° C.

4. The process of claim 1, wherein an amount of water in the liquid product stream is less than 1 wt %.

5. The process of claim 1, wherein the light oil stream further comprises aromatic compounds and paraffins.

6. The process of claim 1, wherein an amount of olefins in the light oil stream is greater than 25 wt %.

7. The process of claim 1, wherein an amount of coke in the heavy oil product stream is less than 1 wt %.

8. The process of claim 1, wherein a weight ratio of the light stream to the light oil stream is in the range between 5:5 and 9:1.

9. The process of claim 2, wherein a temperature of the supercritical water reactor is between 380° C. and 450° C.

10. A system for producing aromatic compounds in a heavy oil product stream, wherein the system comprises:
a flash column, the flash column configured to separate a depressurized effluent to produce a vapor product stream and a liquid product stream;
a vapor cooler fluidly connected to a flash column, the flash column configured to reduce a temperature of the vapor product stream to produce a cooled vapor product;
a three-phase separator fluidly connected to a vapor cooler, the three-phase separator configured to separate the cooled vapor product to produce a light oil stream, wherein the light oil stream comprises 1-olefins;
a splitter fluidly connected to the three-phase separator, the splitter configured to separate the light oil stream to produce a light oil slip stream and a light stream;
a light stream pump fluidly connected to the splitter, the light stream pump configured to increase a pressure of the light stream to produce a pressurized light stream;
a water mixer fluidly connected to the light stream pump, the water mixer configured to mix the pressurized light stream with a pressurized water feed to produce an olefin-containing water stream;
a water pre-heater fluidly connected to the water mixer, the water pre-heater configured to increase a temperature of the olefin-containing water stream to produce a hot water feed, wherein a temperature of the hot water feed is greater than 450° C. such that olefins in the olefin-containing water stream are converted to aromatic compounds;
an oil-water separator fluidly connected to the flash column, the oil-water separator configured to separate the liquid product stream to produce a water stream and a heavy stream; and
an oil mixer fluidly connected to the oil-water separator and the splitter, the oil mixer configured to mix the heavy stream with the light oil slip stream to produce the heavy oil product stream.

11. The system of claim 10, further comprising:
an oil pump, the oil pump configured to increase a pressure of a hydrocarbon feed to produce a pressurized hydrocarbon feed;
an oil pre-heater fluidly connected to the oil pump, the oil pre-heater configured to increase a temperature of the pressurized hydrocarbon feed to produce a hot hydrocarbon feed;
a water pump, the water pump configured to increase a pressure of a water feed stream to produce the pressurized water feed;
a mixer fluidly connected to the oil pre-heater and the water pre-heater, the mixer configured to mix the hot water feed and the hot hydrocarbon feed to produce a mixed feed stream;
a supercritical water reactor fluidly connected to the mixer, the supercritical water reactor configured to maintain conversion reactions to produce a reaction effluent;
a cooling device fluidly connected to the supercritical water reactor, the cooling device configured to reduce a temperature of the reaction effluent to produce a cooled effluent; and
a depressurizing device fluidly connected to the cooling device, the depressurizing device configured to reduce a pressure of the cooled effluent to produce the depressurized effluent.

12. The system of claim 10, wherein a ratio of length to diameter of the flash column is in the range between 5 and 15.

13. The system of claim 10, wherein the temperature of the hot water feed is between 450° C. and 600° C.

14. The system of claim 10, wherein an amount of water in the liquid product stream is less than 1 wt %.

15. The system of claim 10, wherein the light oil stream further comprises aromatic compounds and paraffins.

16. The system of claim 10, wherein an amount of olefins in the light oil stream is greater than 25 wt %.

17. The system of claim 10, wherein an amount of coke in the heavy oil product stream is less than 1 wt %.

18. The system of claim 10, wherein a weight ratio of the light stream to the light oil stream is in the range between 5:5 and 9:1.

19. The system of claim 11, wherein supercritical water reactor comprises one or more tubular reactors with a length to diameter ratio of greater than 100 oriented vertically.

20. The system of claim 11, wherein a temperature of the supercritical water reactor is between 380° C. and 450° C.
